(19) United States
(12) Patent Application Publication
(10) Pub. No.: US 2006/0047309 A1
(43) Pub. Date: Mar. 2, 2006

(54) Metal injection molded suture needles

(76) Inventor: Frank R. Cichocki JR., Easton, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/925,720

(22) Filed: Aug. 25, 2004

(51) Int. Cl. A61B 17/06

(52) U.S. Cl. 606/222

(57) ABSTRACT

The metal-injection molding (MIM) process offers distinct advantages over conventional wire-based methods for producing suture needles. Methods for producing unique suture receiving holes that accommodate large diameter sutures and facilitate adhesive attachment of said sutures are described herein. Additionally, methods for producing cutting edge suture needles that exhibit exemplary tissue penetration performance are described. Finally, the ductility of suture needles produced via the MIM process have been enhanced substantially by employing processes that reduce the internal porosity of the suture needle component.

### FIELD OF INVENTION

The invention pertains to suture needles commonly used to guide and place sutures about a surgical wound. More specifically, the invention pertains to suture needles produced via the metal injection molding process. Novel design features facilitated by the metal injection molding process and methods for improving suture needle properties are disclosed.

### BACKGROUND OF INVENTION

Several researchers have recognized the benefits associated with the ability to attach large diameter sutures to smaller diameter suture needles. The potential benefits include: less tissue trauma from the smaller suture needles, less force required to pass smaller needles through tissue, and enhanced hemostasis at the hole formed in the tissue by virtue of the larger suture plugging the smaller hole left behind by the needle. Matsutani et al. describe a method in U.S. Pat. No. 4,501,312 wherein the proximal end of a suture needle produced from wire is hot forged with a mandrel to produce a suture receiving hole with a diameter that is greater than the diameter of the needle body. Kohut describes a method in U.S. Pat. No. 2,620,028 wherein the entire length of wire to be used to form the suture needle, with the exception of the proximal end, is reduced in diameter by a swaging process. A suture receiving hole is subsequently drilled in the larger proximal end to accommodate a suture. Coplan describes an alternate approach in U.S. Pat. No. 3,918,455 wherein the bore of a hollow monofilament suture is fitted over the proximal end of a suture needle that exhibits a substantially smaller diameter than that of the needle body. A variety of derivations of this concept, wherein heat-shrink tubing has been used to make the connection between a suture and the reduced proximal end of the suture needle, have been disclosed in U.S. Pat. No. 5,226,912, U.S. Pat. No. 5,358,498, and U.S. Pat. No. 5,306,288. However, in all of these examples, in order to accomplish attachment of a large diameter suture to suture needles produced from wire stock, additional, and often costly or time intensive processing steps are necessary.

To prevent unintentional bending and breakage during use, it is desirable to produce suture needles that exhibit exceptionally high strength and ductility. In order to meet these needs, methods of forming needle bodies into the shape of an I-beam have been developed. The I-beam provides excellent bending strength due to the high moment of inertia associated with its shape. Indeed, this simple concept is employed in almost all building structures to produce beams with high bending strength while using a minimum amount of material. Sardelis et. al, in U.S. Pat. No. 5,269,806, claim a needle design and propose a method for producing suture needles with a predominantly rectangular cross-section. The process involves pressing the needle between a series of flat parallel platens. In a first step, the round wire is pressed to form two flat parallel sides. The wire is then rotated 90 degrees and pressed again to produce a needle body with a predominantly rectangular shape with rounded corners, commonly referred to as a rounded I-beam. Matsutani et al. describe a process in U.S. Pat. No. 6,322,581 for producing a hollow I-beam shape wherein the needle is pressed between dies that leave a concave impression on two parallel sides of the needle body, resulting in theory, in even higher needle strengths. Both of the aforementioned techniques for producing strong needle bodies involve additional steps, additional tooling, precision equipment and additional set up time.

Moreover, the stainless steel from which suture needles are commonly produced can be overworked in the process of forming the I-beam, resulting in embrittlement or even splitting of the wire from which the needle is made. Furthermore, in many needle forming processes, the wire is often received in a hardened state making it exceptionally difficult to form into irregular shapes such as an I-beam.

Additional processes are often required to produce a commercially acceptable suture needle. For example, electropolishing processes are commonly used to eliminate flash, splinters, and other surface imperfections that form during the wire forming steps [as described in U.S. Pat. No. 5,269,806]. With certain needle designs, cutting point needles in particular, considerable flash may remain around the needle tip after needle forming and substantial electropolishing can be required to eliminate said flash. However, to optimize the cutting and penetrating performance of the suture needle through tissue, the duration of the electropolishing process and the extent of material removal from the needle should be precisely controlled. If too little material is removed, flash and surface imperfections remain, but if too much material is removed, the cutting edges of the needle may be dulled. While adding to the overall uniformity of the needle, the electropolishing process may detract from the performance of the needle.

The metal injection molding (MIM) process is commonly used to precisely manufacture small metal components that exhibit complicated or unusual shapes. The basic MIM process involves: 1) the injection molding of a feedstock comprised of fine metal powders mixed with a polymeric binder, 2) a debinding step wherein the polymeric binder is removed from the component, and 3) a sintering step wherein the porosity of the component is reduced. However, MIM components are often considered to exhibit mechanical properties that are inferior to the properties that are attainable from components produced via machining operations. Indeed the MIM process is often considered an inferior method for producing surgical devices when excellent mechanical performance is required. To this point, Vecsey et al., in U.S. Pat. No. 5,640,874, have disclosed a method of producing a nominally straight needle that can be used for laproscopic suturing, referred to as a surgical incision member, sharpened on both ends with a suture attached at its center. A variety of methods were described for the manufacture of this component. Among them, MIM was specifically mentioned as a method that was not preferred due to the perception that substandard mechanical properties would result.

Contrary to the teaching of the prior art, however, it has been determined that the MIM process offers an alternate, viable means for producing suture needles with an exemplary combination of strength and toughness with shapes and designs that are not easily produced via the conventional wire forming processes. Near net shape needles that exhibit desirable design features such as large diameter suture receiving holes in the proximal end, I-beam body shape and sharp cutting edges are easily produced. Moreover, all of the needle features, including those that define the point, body, proximal end, and suture receiving hole of the needle, may be produced in a single molding step. Difficult to machine metals that offer excellent materials properties, such as martensitic-aged stainless steels, may be molded into the form of a suture needle via MIM. Finally, a hot isostatic pressing operation may be combined with the MIM process in the manufacture of suture needles to achieve an exemplary combination of strength and ductility.

### SUMMARY OF INVENTION

An embodiment described herein is a method of making a suture needle having two or more cutting edges at a distal portion, comprising the steps of injecting a metal powder feedstock into a mold having at least one parting line, to obtain the suture needle wherein each cutting edges at the distal portion of the needle coincides with a parting line of the mold; and reducing the internal porosity of the suture needle to about 5 percent or less.

An embodiment described herein is a suture needle comprising two or more cutting edges at a distal portion and exhibiting about 5 percent internal porosity or less, that is produced by a process comprising the step of injecting a metal powder feedstock into a mold having at least one parting line, wherein each cutting edge at the distal portion of the suture needle coincides with a parting line of the mold.

Another embodiment described herein is a suture needle having a longitudinal axis comprising a distal portion; a needle body having one or more cross-sectional areas; and a proximal portion; wherein the proximal portion has an outer surface and an inner surface that is coaxial with the outer surface, the inner surface defining the boundary of a suture receiving hole having a cross-sectional area that is greater than or equal to the cross-sectional area of the needle body, and the proximal portion has at least one vent hole that extends from the inner surface to the outer surface such that the suture receiving hole is in fluid communication with the vent hole.

### BRIEF DESCRIPTION OF THE DRAWINGS

FIG. 1 is a schematic representation of a suture needle produced via metal injection molding that exhibits novel design features.

FIG. 2a and b are schematic representations of the mating mold components used to mold the needle shown in FIG. 1.

FIG. 3 is a magnified schematic representation of the mold components shown in FIG. 2 with one of the slide components removed to facilitate viewing.

FIG. 4 is a schematic representation of the proximal end of the suture needle shown in FIG. 1.

FIG. 5. is a schematic representation of the needle body taken through plane A-B-C-D of FIG. 1

FIG. 6a, b, and c are schematic representations of the molding components used to form the suture receiving hole and vent holes.

FIG. 7 is a schematic representation of a cutting point suture needle produced via metal injection molding.

FIG. 8a and b is a schematic cross-sectional representation of the distal portion of the needle shown in FIG. 7 along region A-B-C-D.

FIG. 9a and b, are schematic representations of the distal end and cross-sectional profile of the needle body respectively.

FIG. 10a and b are schematic representations of the mating mold components used to mold the needle shown in FIG. 7.

FIG. 11 is a magnified schematic representation of the mold components of FIG. 9 with one of the mold components removed to facilitate viewing.

FIG. 12a and b are optical micrographs of a suture needle produced via MIM showing the influence of processing on the elimination of internal porosity.

FIG. 13 is a graph of the bending moment applied to the suture needles versus the angle that the curved needle has been rotated through.

FIG. 14 is a graph of the penetration performance of the suture needles versus the penetration pass number.

FIG. 15a and b are photographs of the holes produced in a polymeric media, Porvair, by the impalement and subsequent removal of the suture needles.

### DETAILED DESCRIPTION

As with many other types of surgical devices, suture needles should exhibit exceptional mechanical properties and be able to withstand considerable abuse. It is not uncommon for surgeons to bend and shape suture needles with surgical instruments as they see fit. To test the quality of suture needles they are often plastically deformed through a bend angle of 90 degrees. If the needle does not break in this process it may be deemed to have suitable ductility.

Preliminary investigations indicated that MIM suture needles that had been sintered and heat treated did not offer the high level of ductility available in conventional suture needles produced from wire. Indeed, if MIM needles produced from martensitic or martensitic-aged stainless steel were processed via heat treating to improve the strength of the needle to meet the required strength criteria, ductility was deemed to be deficient. Through further investigation, it was determined that internal porosity, an artifact of incomplete sintering and densification, inhibited the MIM suture needles from exhibiting an exemplary combination of ductility and strength. It was further determined that if the internal porosity of the MIM suture needle can be reduced to less than 5 percent by volume, preferably to less than 3 percent by volume, and more preferably to less than 1 percent by volume, the needles may exhibit the requisite high ductility and strength, making them competitive with conventional suture needles produced from wire. It was determined that a process commonly referred to as hot isostatic pressing may be used to reduce the porosity contained in a MIM suture needle to as low as about 1 percent or less by volume. The improvement in ductility becomes apparent, with the MIM suture needles easily meeting the 90 degree reshape requirement at strength levels that are competitive with commercially available suture needle produced from wire, as measured according to ASTM standard F1874-98 (reapproved 2004).

As discussed above, the MIM process involves: 1) the injection molding of a feedstock comprised of fine metal powders mixed with a polymeric binder, 2) a debinding step wherein the polymeric binder is removed from the component, and 3) a sintering step wherein the porosity of the component is reduced. Injection molding temperatures and pressures may vary widely depending on the feedstock characteristics, mold cavity design, and part size. Typical injection pressures and temperatures typically fall in the range of 100 to 2500 bar and 150 to 250° C. respectively. Debinding procedures vary widely with the type of binder used in the feedstock material but may include: pyrolysis of a polymeric binder at temperatures ranging from 50 to 600° C., catalytic debinding of the polymer binder wherein a reactive gas assists degradation and removal of the binder, and solvent removal of the binder wherein the molded part is exposed to a solvent that dissolves and removes a majority of the binder. Sintering processes likewise vary widely according to the metal powder composition, particle size, distribution, and particle morphology.

The internal porosity of the suture needle produced via MIM may be reduced to less than about 5 percent by volume, preferably to less than about 3 percent by volume, and more preferably to about 1 percent by volume or less by any known method, including but not limited to hot isostatic pressing. Generally, hot isostatic pressing is described in the ASTM Handbook, Volume 7 Powder Metal Technologies and Applications.

The MIM process offers several advantages over conventional wire forming processes for the manufacture of suture needles. Firstly, since the shape of a MIM suture needle is proportional to the shape of the mold used to produce the needle, the intricacy of the needle design is limited for the most part only by the design of the mold. To this point, molds used in the MIM process are often produced using computer aided 3-D machining processes, such as electro-discharge machining, EDM, that offer excellent three-dimensional design flexibility. Moreover, where limitations exist, a novel mold-slide configuration may be employed to overcome such limitations.

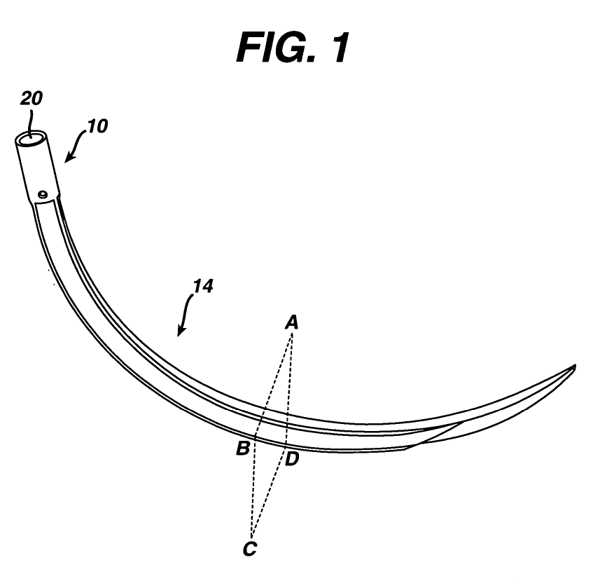

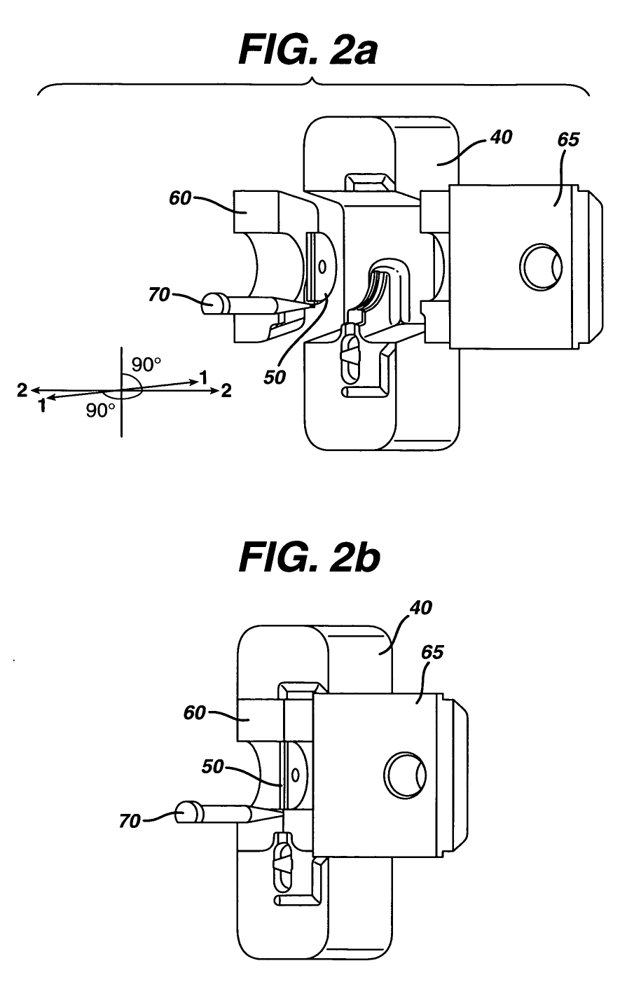

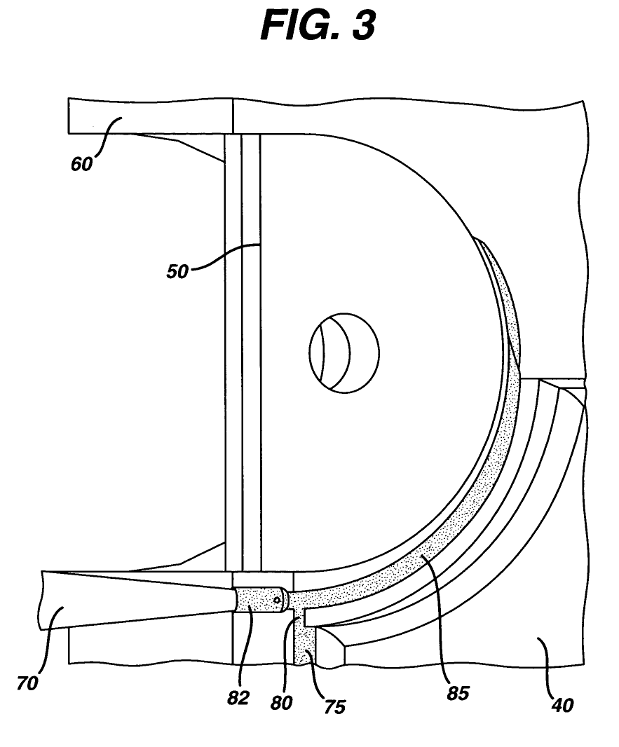

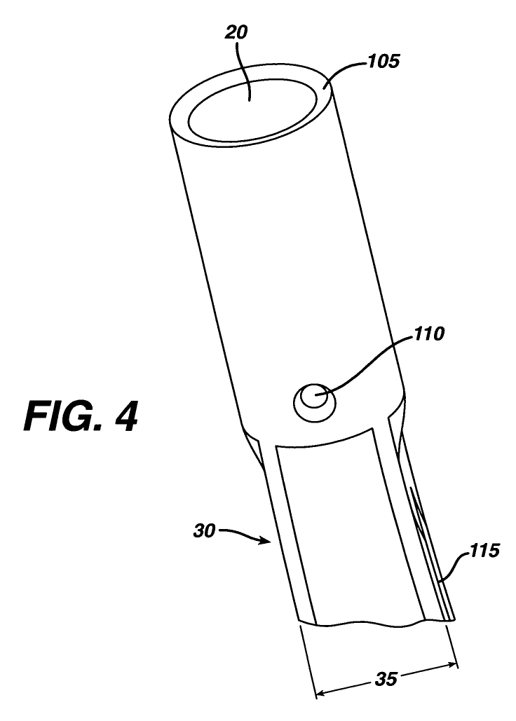

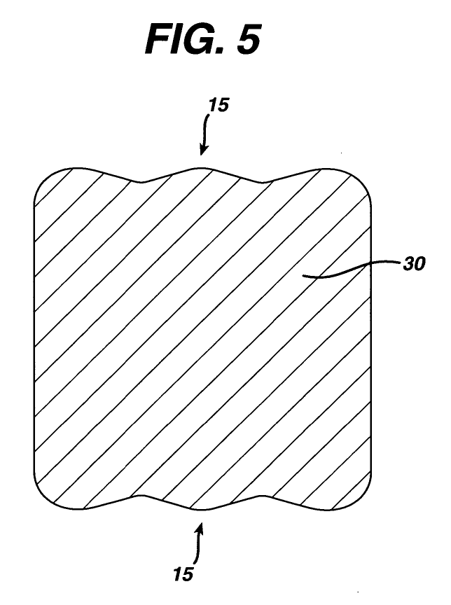

For example, as schematically represented in FIG. 1, needles that exhibit a proximal end 10 with a suture receiving hole 20 that is approximately equal to or larger in cross-sectional area than that of the needle body 14 may be produced via MIM. The mold components that form the shape of the needle, shown in an expanded view in FIG. 2a, include: a core 40, insert 50, two slides 60 and 65 and a core pin 70. The slides 60, 65, core pin 70, and insert 50 may move one after the other, in pairs, or in unison along the directions 1-1 and 2-2 indicated in FIG. 2a. The needle is molded when the mating components of the mold are in the fully closed position as shown in FIG. 2b. A magnified view of this configuration, minus the slide component 65, is shown in FIG. 3. A molten feedstock 75, comprised of metal powders and a polymeric binder, is injected through the gate 80, thereafter filling the space 82 around the core pin 70 and the remainder of the mold cavity 85. The slide 60 is then retracted to the open position, shown in FIG. 2a, and the insert slide 65 is moved forward, so the needle can be extracted from core pin 70 and the mold. The core pin 70, which forms the inside wall of the suture receiving hole 20 shown FIG. 4, may possess a diameter that is substantially equal to or greater than the diameter of the needle body 30 and the two slides 60 and 65 may be designed to close concentrically around the core pin leaving a small space to accept feedstock and form the wall 105 of the suture receiving hole. In this way, a needle with a proximal end and suture receiving hole that is substantially equal to or greater in cross-sectional area than that of the needle body may be produced. The large diameter suture receiving hole 20 schematically represented in FIG. 4 can accommodate a suture that approaches or exceeds the diameter of needle body 30. Conventional swaging processes, commonly employed in the art of suture making, may be used to simultaneously attach the suture to the proximal end of the needle and reduce the cross-section of the proximal end to a size and shape coincident with the dimensions of the needle body. For a given suture size, benefits of this design include: less tissue trauma associated with passage of smaller needles, less force required to pass smaller needles through tissue, and enhanced hemostasis at the hole produced by the suture needle by virtue of the larger suture plugging the smaller hole left behind by the needle.

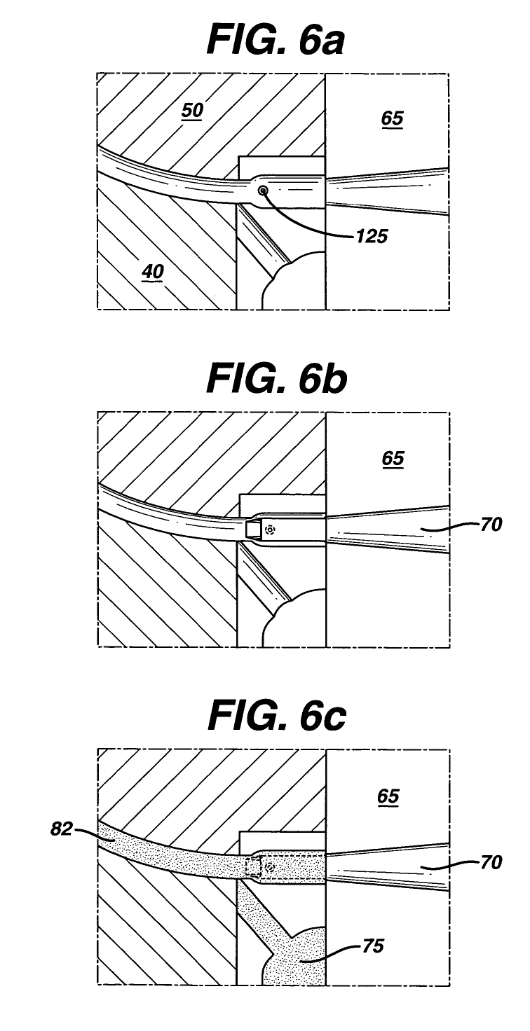

In addition to facilitating the attachment of larger diameter sutures to smaller diameter needles, the MIM process may be used to produce suture needles with features that facilitate the adhesive attachment of sutures. One problem associated with the adhesive attachment of sutures to suture needles, especially with a viscous adhesive, is the entrapment of air within the suture receiving hole located in the proximal end of the suture needle. The adhesive may be placed directly onto the suture prior to insertion into the suture receiving hole, or may be first injected into a portion of the suture receiving hole directly. In either case, as the suture is inserted into the suture receiving hole, air compresses and produces a counter-pressure that either opposes complete insertion of the suture or forces the suture back out of the hole over time before the adhesive cures. One or more vent holes 110 at the base of the suture receiving hole 20, schematically depicted in FIG. 4, would allow the air to exhaust thereby enabling insertion of the suture into the needle without the risk of it being forced back out by trapped compressed air. As a further benefit, the adhesive may partially fill and mechanically interlock with the vent hole 110 thereby increasing the overall force required to detach the suture from the needle. When producing suture needles with the MIM process, these vent holes may be molded into the suture needle in a single molding operation. For example, as schematically represented in FIG. 6a, a protrusion 125 may be formed in the slides 60 and/or 65. As the mold closes and the core pin 70 used to form the suture receiving hole is inserted, as shown in FIG. 6b, the protrusion 125 mates against the core pin 70. Feedstock 75 is molded around both the core pin and protrusion, as shown in FIG. 6c, so that when the suture needle is ejected from the mold, one or more vent holes 110 are produced at the base of the suture receiving hole 20, as shown in FIG. 4. The size, shape and location of the vent holes along the proximal end of the suture needle may vary to meet performance needs. Typically, the vent holes would be located in the distal two-thirds of the suture receiving hole in order to enable deep insertion of the suture. Other practical limitations exist. For example, the size of the diameter of the vent holes cannot exceed the diameter of the suture receiving hole. Vent holes need not exhibit circular profiles, but may alternatively exhibit elongate or polygonal profiles.

The mold components presented in FIGS. 2 and 3 may also produce optional features on the inside and outside curved surfaces of the needle. For example, FIG. 5 shows a cross-sectional view of a needle body taken along slice A-B-C-D in FIG. 1, where ribs 15 that travel longitudinally along the needle body 14 may be produced on the opposing sides of the needle body.

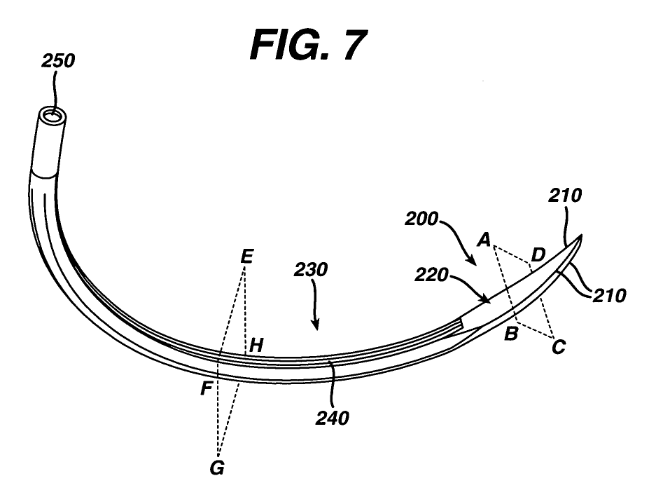

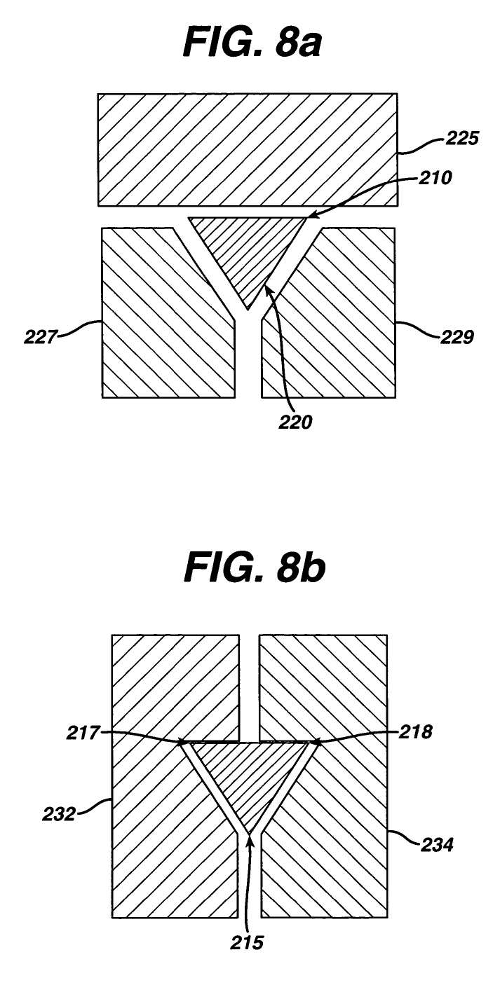

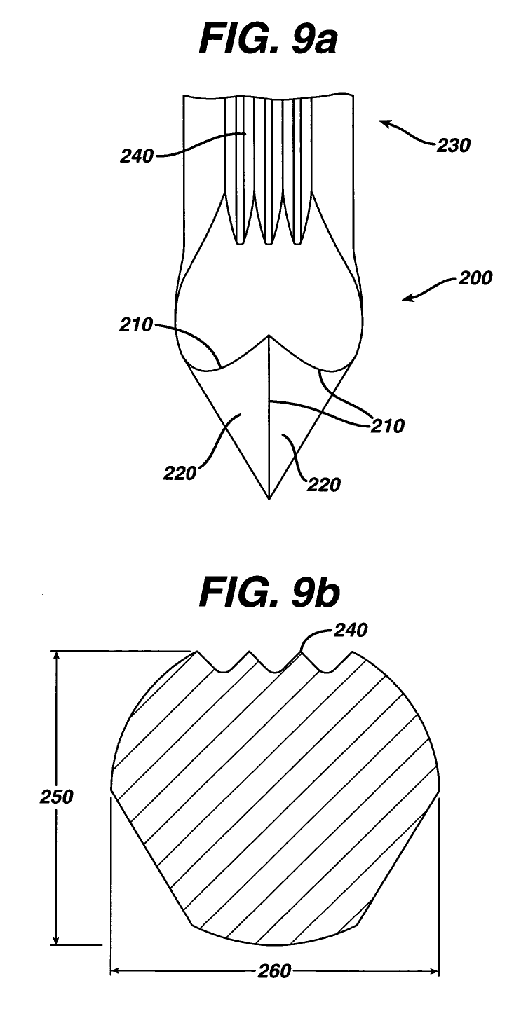

Standard cutting point suture needles such as the design schematically depicted in FIG. 7 may also be produced via MIM. These MIM suture needles may be made to exhibit penetration performance superior to their wire-machined counterparts. A novel mold-slide configuration may be employed to produce sharp, flash-free cutting edges 210 along the distal end 200 of the needle 230, where each cutting edge coincides with a parting line of the mold. A parting line as used herein corresponds to the line formed at the junction of two molding components and the molded part. For example in a simple configuration that employs two opposing mold halves, a single parting line may be formed around the circumference of the molded part at the location where the mold halves came together. In a more complicated configuration employing three mold components to produce a part, three parting lines may be formed where the three discrete molding components contact one another and the molded part. A cross-sectional representation of the distal portion of the MIM suture needle taken along region A-B-C-D of FIG. 7 is shown in FIG. 8a along with a cross-sectional view of the mold components that may be used to produce the suture needle. The distal end of the needle is depicted in cross-section in FIG. 8a and in profile view in FIG. 9a as triangular in cross-section with 3 cutting edges 210 and three faces 220. With the mold-slide configuration of FIG. 8a, each of the cutting edges of the needle is coincident with a parting line of the mold components 225, 227, and 229. Suture needles having two, three or four cutting edges may be made as described above if each cutting edge is made to coincide with a parting line of the mold. Since the MIM needles produced in this way exhibit sharp edges with little to no flash, an abbreviated electropolishing step of about 30 seconds or less is sufficient to finish the needles and the sharp cutting edges are retained along the distal cutting edges of the needles. Good penetration performance also results.

It is important to note that the aforementioned mold-slide configuration is not intuitive and a simpler design employing only two molding halves, as schematically represented in FIG. 8b, may have otherwise been employed. Indeed, fewer molding components and mold machining operations are associated with the mold design of FIG. 8b. However, this molding configuration does not lend itself to producing sharp cutting edges along the entire distal portion of the needle. Indeed with this design, only one cutting edge 215 is coincident with the parting line of the mold, while the other two cutting edges 217 and 218 of the needle are formed within the cavities of the opposing mold halves 232 and 234. The sharpness of these latter two cutting edges is consequently limited by the resolution of the machining process used to form the mold halves, which is inevitably less sharp than the sharp edge formed by the junction of the independent mold components.

In addition to novel suture needle designs facilitated by the MIM process, certain metal alloys that offer favorable performance may be produced via MIM. For example, martensitic stainless steels, such as 420 grade, with a nominal composition of 12 to 14% chromium, 0.1 to 0.4% carbon with the balance being iron, may be utilized. The group of steels classified as martensitic-aged or mar-aged steels provides another prime example. 17-4 grade martensitic-aged, or mar-aged, stainless steel, commonly used in the MIM process and quite suitable as a material for suture needles, typically has a nominal composition of 15 to 17.5% Cr, 3 to 5% Ni, 3 to 5% Cu and less than 1% Si with the balance being iron. Other martensitic-aged steels, such as those disclosed in U.S. Pat. No. 5,000,912 and U.S. Pat. No. 5,651,843 for the explicit use as a materials for suture needles, with nominal compositions of 12 to 14% Cr, 7 to 11% Ni, and 1 to 2.5% Ti with the balance comprising iron, may also be considered as good candidate materials for producing suture needles via the MIM process. These alloys exhibit properties that are desirable in a suture needle, such as high strength, toughness, and stiffness.

Other alloys, such as those that exhibit high hardness, may offer a high level of resistance to the damage that is commonly incurred during processing or surgical use of suture needles. However, as the hardness of the metal alloy approaches the hardness of the tools that are used in the wire forming process, it becomes difficult and costly to produce the suture needle. Moreover, conventional wire forming processes will not allow investigation of hard materials, such as carbides or ceramics, for the production of suture needles since these materials cannot be formed into a ductile wire. The MIM process on the other hand may be used to produce components from most materials that may be reduced to the powder feedstock, including a multitude of metal alloys, carbides, and ceramics. The list of alternate materials that may be easily manufactured into the form of a suture needle via the MIM process include but are not limited to: carbide materials such as tungsten carbide cobalt cermets, a variety of ceramics including aluminum oxide, silicon nitride, silicon carbide, and titanium carbide, tool steels, mar-aged stainless steels, martensitic steels, and titanium alloys. Particle sizes, particle morphologies, and particle size distributions of the metal powders in the feedstock material are highly variable from one feedstock material to the next. Typical particle sizes may range from sub-micrometer up to 200 μm, and preferably from ˜4 to ˜50 μm. Moreover particles may exhibit considerable asymmetry.

### EXAMPLE 1

The needle that is schematically depicted in FIG. 1 was produced via metal injection molding. This needle has radiused or “undercut” cutting edge described by U.S. Pat. No. 5,797,961. The body portion is not round in cross-section but is rather rectangular with average dimensions of 0.0245″ by 0.0240″. In addition, as shown in FIG. 5, ribs have been molded into the inside and outside curvatures of the needle body. The proximal end of the needle shaft has an outside diameter of 0.030″ with an inside diameter of 0.024″ making it compatible with a size 2 suture according to United States Pharmacopia, USP, standards. The mold components used to produce this needle are shown in FIGS. 2, 3 and 6. Needles were molded from two different feedstock materials. The first feedstock contained the constituent powders of a martensitic stainless steel, commonly referred to as 420 grade. The other feedstock contained the constituent powders of a matensitic-aged, or mar-aged, stainless steel, commonly referred to as 17-4. In both samplings, approximately 60 volume percent of the feedstock was comprised of metal powders, nominally less than 20 □m in diameter, with the remainder being comprised of a polyacetal polymeric binder. The feedstock was injected into the mold that is schematically depicted in FIG. 2 with an injection pressure of 1715 bar, at a feedstock temperature of 204° C., with a mold temperature of 121° C., at a cycle time of 15 to 20 seconds. Molded components were ejected from the mold and collected on alumina substrates. A catalytic debind process, as described in U.S. Pat. No. 4,624,812 and U.S. Pat. No. 5,531,958 followed, wherein the samples were heated to ˜150° C. under a nitrogen gas mixture containing nitric acid to remove the majority of the polymeric binder. The needles produced from the 420 constituent powders were sintered at 1335° C. for 10.5 hr in argon gas at atmospheric pressure. The needles produced from 17-4 constituent powders were sintered at 1290° C. for 12.5 hr in a hydrogen gas environment. Hot isostatic pressing was then conducted in an argon environment at 104 MPa at 1100° C. for 3 hours. All needles were subsequently heat treated, electropolished, and siliconized with processes well-known in the art. Needles produced from 420 stainless steel were subjected to a heat treatment that involved an air-quench from 1020° C. to room temperature and subsequent cooling to −196° C. by immersion in liquid nitrogen to produce a fully martensitic structure. A tempering process was then conducted at 420° C. for 20 minutes to attain a good combination of strength and ductility. Needles produced from 17-4 stainless steel were air-quenched from 1050° C. to room temperature and subsequently subjected to a precipitation or aging treatment at 450° C. for 30 minutes.

### EXAMPLE 2

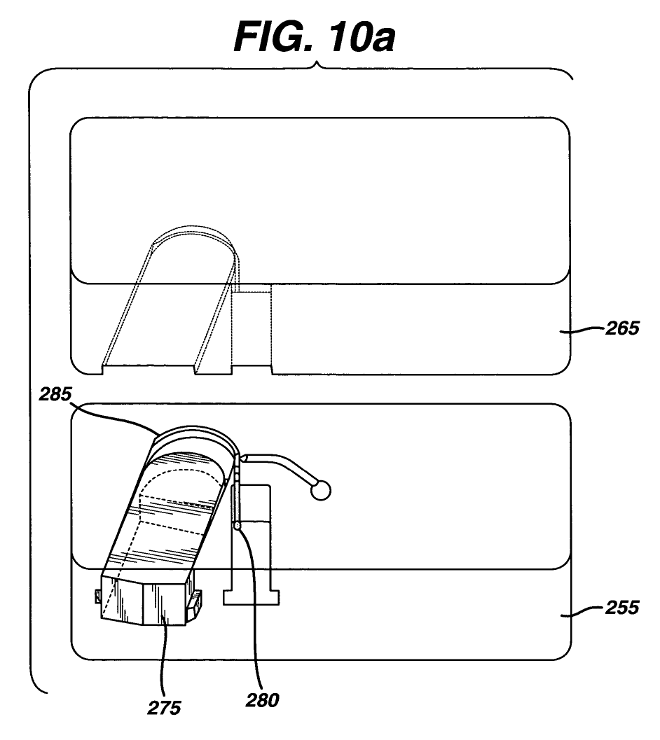

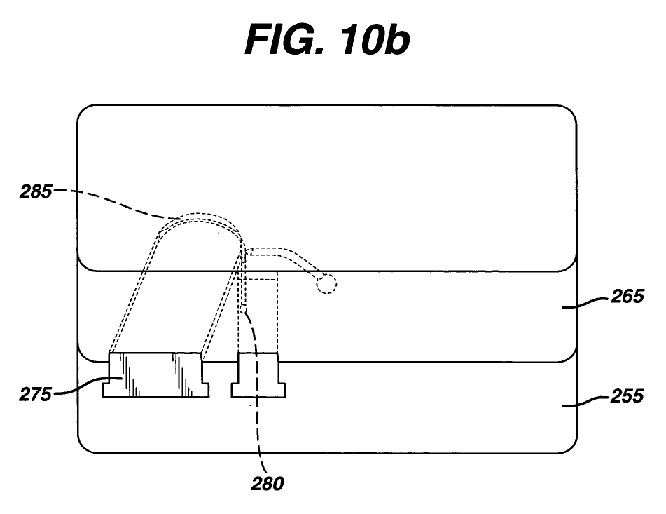

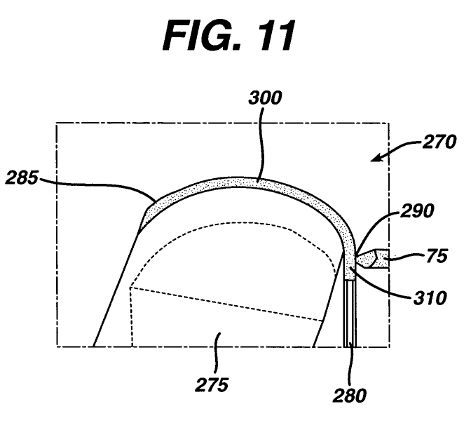

The suture needle schematically depicted in FIG. 7 was produced via MIM. This suture needle exhibits a standard cutting tip with three cutting edges 210 and flat faces 220 that taper to a point from the needle body, as shown in FIG. 9a. The needle body has ribs 240 as schematically depicted in FIG. 9b, which represents the cross-sectional view taken along E-F-G-H in FIG. 7. The height 250 and width 260 of the needle body were 0.0392″ and 0.0416″ respectively. The suture receiving hole 250 in FIG. 7, was 0.0060″ deep with an inside diameter of 0.0202″ and was able to accommodate a size 0 and 2-0 suture, referring USP standards. The four mold components used to form this needle, a core 255 and cavity 265, slide 275 and core pin 280, are shown in FIG. 10a in the open position. The mold components move from the open position schematically depicted in FIG. 10 a to closed position shown in FIG. 10b. A close-up view of the mold with the suture needle 285 included is shown in FIG. 11. The cavity component 265 has been hidden to provide a clear view. Feedstock material 75 flowed through the gate 290 and into the mold cavity 300 including the space surrounding the core pin 310 to form a suture receiving hole. This needle was produced with both the martensitic stainless steel, 420 grade, as well as with the martensitic-aged stainless steel referred to as 17-4. The feedstock material was forced into the mold at an injection pressure of 610 bar, otherwise, the same processing parameters described in Example 1 were employed.

### EXAMPLE 3

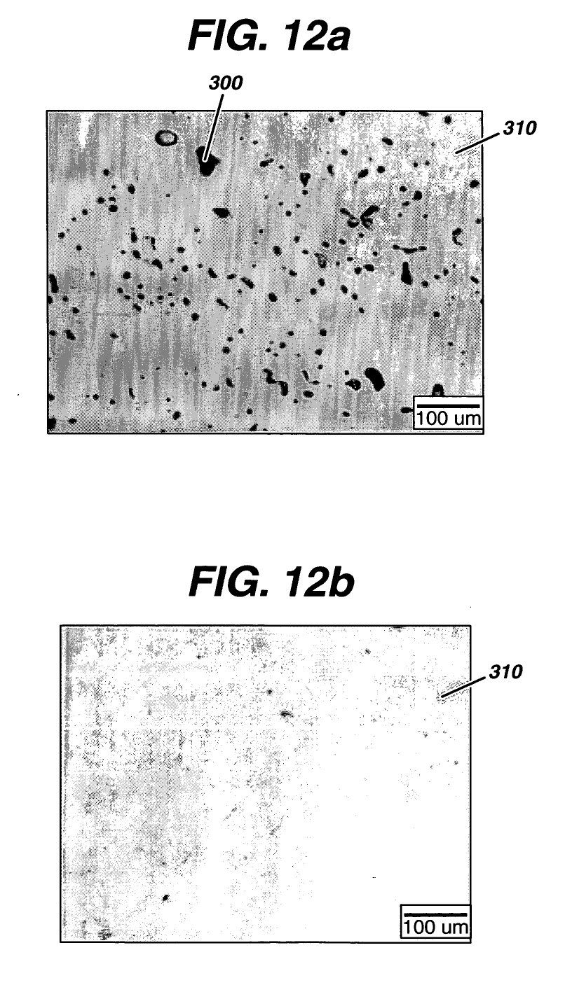

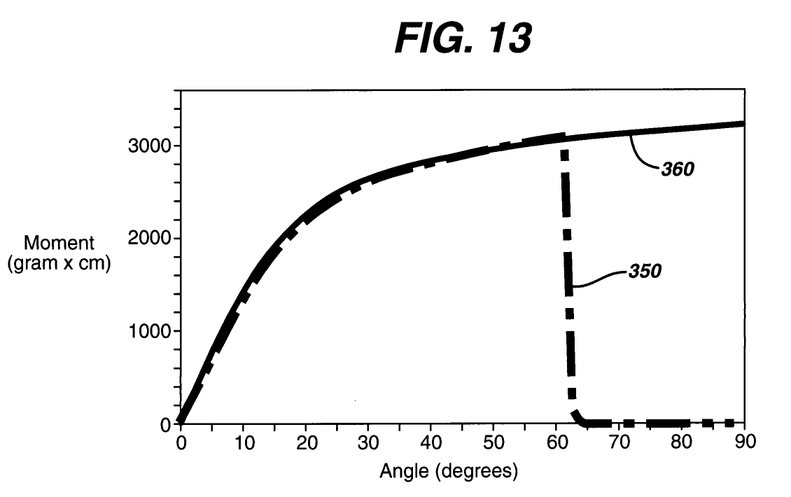

Suture needles produced from 420 stainless steel feedstock under the processing parameters described in Example 2 exhibited up to 6 volume percent internal porosity after the sintering process. A micrograph taken of the MIM needle described in Example 2 after the sintering process, but before a hot isostatic pressing process is shown in FIG. 12a. The black phase 300 is porosity and the light phase 310 is dense metal. The pores are evenly dispersed, ranging in size from a few micrometers to ˜50 micrometers. After subjecting the needles to a hot isostatic pressing process, wherein they were processed at 1100° C. for 3 hours under 104 MPa of gas pressure in an argon environment, the porosity was reduced to less than 1 volume percent. As shown in the optical micrograph of FIG. 12b, no dark phase is detectable indicating that the porosity was essentially eliminated. An improvement in ductility coincided with the decrease in internal porosity. As indicated by the dashed curve 350 in FIG. 13, needles that contain internal porosity on the order of 5 to 6 volume percent (corresponding to the needles shown in the micrograph of FIG. 12a) fractured when the needle body was bent through an angle of ˜65 degrees. Alternatively, needles that were hot isostatically pressed and exhibited less than 1 volume percent internal porosity remained intact after being bent through an angle of 90 degrees, as indicated by curve 360 in FIG. 12.

### EXAMPLE 4

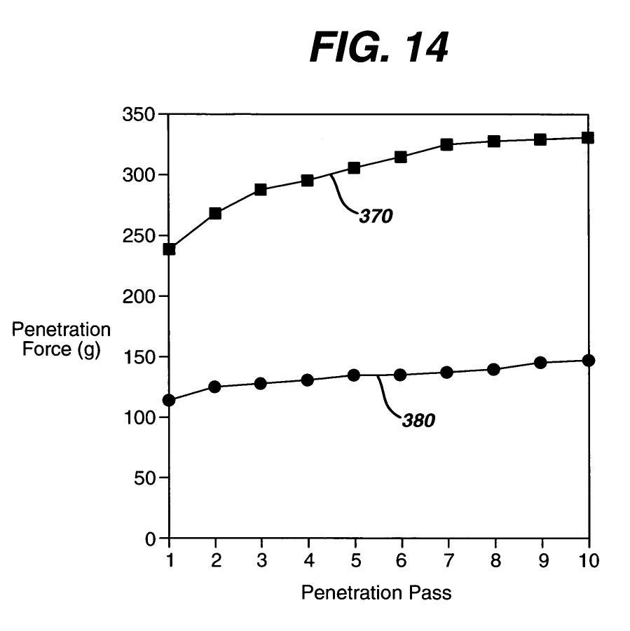

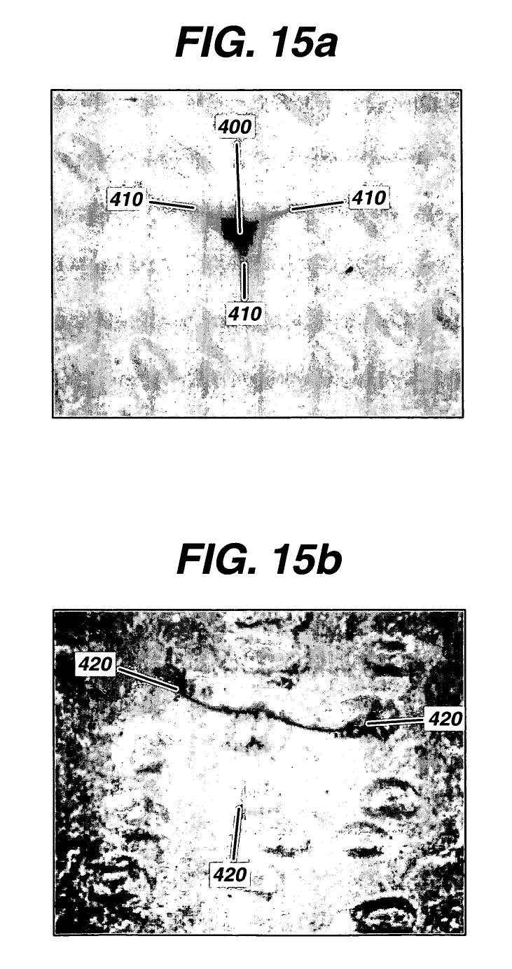

The penetration performance of the MIM needle, produced from alloy 17-4, described in Example 2 and schematically depicted in FIG. 7, was compared to the penetration performance of an Ethicon cutting point needle of the same design produced from wire and designated as CP-1 (a surgical needle commercially available from Ethicon, Inc., located in Somerville, N.J.). The method for evaluating the penetration performance of a needle is described in U.S. Pat. No. 5,181,416, which is hereby incorporated in its entirety. Specifically, both sets of needles were subjected to a brief electropolishing step for 20 seconds with a solution containing phosphoric acid, sulfur acid, glycolic acid, and water to remove any metal slivers, flash, or other surface imperfections. Both sets of needles were siliconized according to methods well-known in the art. The penetration performance was assessed by forcing the needles through a polymeric material having a thickness of 1.1 mm and referred to commercially as Porvair™ while measuring the maximum force required to penetrate said media. This value was referred to as penetration force and the measurement was taken for 10 consecutive passes for each needle tested. The results are presented in FIG. 14 in terms of penetration force versus penetration pass number. Substantially less force, at least about 30 percent less force and up to about 50 percent less force, was required to penetrate the MIM needles through the Porvair™ media than the conventional wire-based CP-1 needles. This result was substantiated by the observation that the MIM needles leave behind different penetration marks in the Porvair™ than the Ethicon CP-1 needles. As shown in FIG. 15a, a puncture hole 400 was left behind by the wire-based CP-1 needle. Three short incisions 410, corresponding to the cutting edges of the needle, are detectable along the perimeter of the puncture hole. In contrast, as shown in FIG. 15b, 3 long incision marks 420, corresponding to the cutting edges of the needle, and virtually no puncture hole was produced by the MIM suture needle. Producing an incision, as shown in FIG. 15b, typically requires less force and material distortion than producing a blunt puncture, as shown in FIG. 15a. The superior penetration performance exhibited by the MIM needles is thus attributed to the retention of sharp cutting edges along the distal portion of the needle. The retention of sharper cutting edges was facilitated by the mold design shown in cross-sectional view in FIG. 8a wherein the parting lines of the mold coincided with the cutting edges of the needle. Additionally, the ability to use abbreviated electropolishing cycles, on the order of 15 seconds to finish the near net-shape MIM needles vs. greater than 60 seconds for wire-based needles, likely contributed to the superior cutting edge retention and penetration performance attained with MIM needles.

What is claimed is:

1. A method of making a suture needle comprising the steps of:
injecting a metal powder feedstock into a mold to obtain the suture needle; and
reducing the internal porosity of the suture needle to about 5 percent or less.

2. The method of claim 1, where the internal porosity of the suture needle to about 3 percent or less.

3. The method of claim 1, where the internal porosity of the suture needle to about 1 percent or less.

4. The method of claim 1, wherein the internal porosity of the suture needle is reduced via hot isostatic pressing

5. A method of making a suture needle having two or more cutting edges at a distal portion, comprising the steps of:
injecting a metal powder feedstock into a mold to obtain the suture needle wherein each cutting edge at the distal portion of the needle coincides with a parting line of the mold; and
reducing the internal porosity of the suture needle to about 5 percent or less.

6. The method of claim 5, where the internal porosity of the suture needle to about 3 percent or less.

7. The method of claim 5, where the internal porosity of the suture needle to about 1 percent or less.

8. The method of claim 5, wherein the internal porosity of the suture needle is reduced via hot isostatic pressing.

9. A suture needle exhibiting about 5 percent internal porosity or less, that is produced by a process comprising the steps of injecting a metal powder feedstock into a mold to obtain the suture needle; and reducing the internal porosity of the suture needle to about 5 percent or less.

10. The suture needle of claim 9, wherein the suture needle exhibits about 3 percent internal porosity or less.

11. The suture needle of claim 9, wherein the suture needle exhibits about 1 percent internal porosity or less.

12. The suture needle of claim 9, further comprising a needle body having one or more cross-sectional areas; and a proximal portion; wherein the proximal portion has an outer surface and an inner surface that is coaxial with the outer surface, the inner surface defining the boundary of a suture receiving hole having a cross-sectional area.

13. The suture needle of claim 12, wherein the cross-sectional area of the suture receiving hole is greater than or equal to the cross sectional area of the needle body.

14. The suture needle of claim 12, further comprising a vent hole in the proximal portion, wherein the vent hole is in fluid communication with the suture receiving hole.

15. A suture needle comprising two or more cutting edges at a distal portion and exhibiting about 5 percent internal porosity or less, that is produced by a process comprising the steps of injecting a metal powder feedstock into a mold to obtain the suture needle wherein each cutting edge at the distal portion of the needle coincides with a parting line of the mold; and reducing the internal porosity of the suture needle to about 5 percent or less.

16. The suture needle of claim 15, wherein the suture needle exhibits about 3 percent internal porosity or less.

17. The suture needle of claim 15, wherein the suture needle exhibits about 1 percent internal porosity or less.

18. The suture needle of claim 15, further comprising a needle body having one or more cross-sectional areas; and a proximal portion; wherein the proximal portion has an outer surface and an inner surface that is coaxial with the outer surface, the inner surface defining the boundary of a suture receiving hole having a cross-sectional area.

19. The suture needle of claim 18, wherein the cross-sectional area of the suture receiving hole is greater than or equal to the cross sectional area of the needle body.

20. The suture needle of claim 18, further comprising a vent hole in the proximal portion, wherein the vent hole is in fluid communication with the suture receiving hole.

21. The suture needle of claim 15, wherein the maximum force required to penetrate a 1.1 mm Porvair™ polymeric material is at least 30 percent less than the maximum force required by the same needle design made from wire.

22. A suture needle having an longitudinal axis comprising
a needle body having one or more cross-sectional areas; and a proximal portion;
wherein the proximal portion has an outer surface and an inner surface that is coaxial with the outer surface,
the inner surface defining the boundary of a suture receiving hole; and
the proximal portion having at least one vent hole that extends from the inner surface to the outer surface such that the suture receiving hole is in fluid communication with the vent hole.
